(19) United States
(12) Patent Application Publication
(10) Pub. No.: US 2011/0111032 A1
(43) Pub. Date: May 12, 2011

(54) MANUFACTURING METHOD OF COLLAGEN GEL COMPOSITION FOR BONE REGENERATION

(75) Inventors: Jae-Deog Jang, Seoul (KR); Hun Kim, Seoul (KR); Ji-Chul Yu, Seoul (KR); Se-Geun Yeo, Gyeonggi-do (KR); Tae-Hyoung Kim, Seoul (KR); Hyun-Shin Park, Seoul (KR); Seon-Ae Kim, Seoul (KR); Jang-Hoon Kim, Seoul (KR); Seong-Soo Kim, Seoul (KR)

(73) Assignee: SEWON CELLONTECH CO., LTD., Seoul (KR)

(21) Appl. No.: 13/003,157

(22) PCT Filed: Jul. 23, 2008
(86) PCT No.: PCT/KR08/04287

§ 371 Date: Jan. 7, 2011

(30) Foreign Application Priority Data
Jul. 11, 2008 (KR) 10-2008-0067692

(51) Int. Cl. A61K 9/14; A61K 35/12; A61P 19/00

(52) U.S. Cl. 424/484; 424/93.7

(57) ABSTRACT

Disclosed herein is a method for preparing a collagen gel composition for bone regeneration comprising collecting bone marrow from animal tissues and isolating nucleated cells from the bone marrow; and mixing the nucleated cells and a bio-matrix composed of type I collagen and apatite.

### TECHNICAL FIELD

The present invention relates to a method for preparing a collagen gel composition for bone regeneration. More specifically, the present invention is capable of promoting osteogenesis by imparting osteoconductivity, osteogenicity and osteoblastic differentiation induction capacity through co-injection of bone marrow-derived nucleated cells with a collagen-based matrix composition into bone-defective lesions of a subject. Further, the present invention can ensure high quality of the product through the process and quality control where a matrix composition can be used for medical applications. In addition, the present invention enables mass production of the product, use of the product alone or in combination with bone marrow-derived nucleated cells of patients, if necessary, and convenient and low-cost application of the product to the patient within a short period of time, as compared to conventional cell therapeutic agents. Therefore, the present invention accomplishes significantly improved quality and reliability of the product and thereby is very useful to enhance customer satisfaction.

### BACKGROUND ART

The present invention is an improvement to Korean Patent Application No. 2006-0091325 (Patent Registration No. 0834718) assigned to the present applicant and entitled “osteogenesis-promoting cell composition for bone regeneration and method for preparing the same”.

As is generally known, osteoporosis is a medical condition that results in a gradual loss of bone mass and density and as a result, is accompanied by high susceptibility to bone fractures resulting from the formation of increased numbers of tiny pores within the bone, similar to that found in coarse pumice stones or sponges. That is, osteoporosis is a disease of progressive bone loss involving the formation of many tiny holes or pores as compared to normal bone, reduction of bone mass, thinning and weakening of bone microarchitecture, thus causing the bones to become brittle and prone to breaking even with only light impact. Because osteoporosis progresses silently without subjective pain or symptoms, people might not be aware that they have osteoporosis until they accidentally fall or get hit and in turn easily break a bone. Even with light falls, people having osteoporosis may experience wrist fractures, pelvic fractures and vertebral fractures with accompanying severe pain. In particular, pelvic fractures and vertebral fractures are severely painful and require surgical operations, and the patients have to put up with the hardship of being sick in bed even for several months. Even after complete recovery from the surgery, physical impairment may still remain due to surgical sequalae or complications of osteoporosis.

Bones continuously undergo decomposition and replacement processes. That is, remodeling occurs constantly in all bones. During bone remodeling, old bone is destroyed and absorbed by osteoclasts, and new bone is formed by osteoblasts. If bone absorption exceeds bone regeneration due to imbalanced homeostasis of the bone tissues, this may lead to the occurrence of osteoporosis. It is known that the incidence of osteoporosis has a relationship with combination of various risk factors such as female gender, a thin and/or small body frame, advanced age, a family medical history of osteoporosis, menopause (including hysterectomy), irregular menstruation (amenorrhoea), neurasthenia, use of adrenocortical hormones or anticonvulsants, hypoandrogenemia in male gender, insufficient exercise, smoking, excessive drinking, Asian and Caucasian people (Africans and Hispanic-Americans are at lower risk), early menopause (before age 45), excessive caffeine and alcohol consumption, and diets low in calcium.

The incidence of osteoporosis is higher in Asian people than American people. Osteoporosis is estimated to affect more than 28,990,000 American people (80 percent of those affected are women). In the United States, 10 million individuals already have osteoporosis. 18 million more have low bone mass, placing them at increased risk for osteoporosis. One in two women and one in eight men among American Caucasians over the age of 50 will experience osteoporosis-related fractures in their lifetime. One in ten African-Americans over the age of 50 has osteoporosis; an additional one in 3 has low bone density that puts them at risk of developing osteoporosis. Osteoporosis is responsible for more than 1.5 million fractures annually, including: 300,000 hip fractures, 700,000 vertebral fractures, 250,000 wrist fractures and 300,000 fractures at other sites. In the United States, 12,000,000 fracture cases occur each year, 147,000 to 250,000 cases of which are hip fractures and 80% of which are caused by light trauma. By age 80, about 40% of women experience at least one vertebral fracture. A third of women and a sixth of men will experience a hip fracture by the time they are in their late 80s. 25 to 50% of the hip fracture patients are unable to walk without the assistance of another person even after hip repair surgery and such fractures are known to be connected with the mortality.

Among various therapeutic approaches developed to treat osteoporosis, conventionally-established osteoporosis therapies, such as by use of bisphosphonates or selective estrogen receptor modulators (SERMs), primarily focus on the suppression of bone absorption and are known to inhibit progress of osteoporosis via the prevention of a further loss of bone mass. In addition, by using bone grafting or autologous osteoblast-based therapeutic agents, bone union or bone regeneration can be achieved in local fractures caused by various factors including osteoporosis or in target lesions requiring bone regeneration.

However, the conventional therapeutic methods block further progress of osteoporosis by preventing bone absorption via inhibition of the osteoclast activity, and therefore suffer from problems related to failure of substantially facilitating bone regeneration. Further, use of the above-mentioned bone graft technique or autologous osteoblast-based therapeutic agents is disadvantageous in that it is difficult to achieve biological bone regeneration throughout extensive regions.

Even though development of cell therapeutic agents has been driven in order to overcome disadvantages of bone regeneration suffered by the classic bone graft techniques, it is difficult to achieve systemic application of adherent cells or therapeutic treatment of adherent cells via the blood stream. This is because it is impossible to carry out cell injection via the blood stream since adherent cells may die if they are not adhered to an adequate substrate.

Reviewing further details of the conventional schemes, therapeutic methods using bone allografting, bone autografting or transplantation of autologous osteoblast-based therapeutic agents for local application have been used when bone defects or osteonecrosis took place in local lesions, or otherwise therapeutic methods using bone-absorption inhibitors such as bisphosphonate and the like have been used when bone defects have occurred throughout a broad range of lesions such as osteoporosis. Bone allografting still suffers from problems such as propagation possibility of diseases, insufficient supply of implant materials, occurrence of undesired immune reaction such as graft rejection, and a difficulty in complete regeneration of the implants into self tissues of a subject. Meanwhile, bone autografting solves or alleviates such problems suffered by the allograft technique, but has disadvantages such as a difficulty of securing sufficient donor sites to provide bone for bone transplantation, the morbidity of the donor sites and the like. A cell therapeutic agent utilizing autologous osteoblasts is a therapeutic approach which was developed to solve the problems and disadvantages of the conventional bone graft techniques, and is known as a technique which is capable of achieving local bone regeneration by mass proliferation of osteoprogenitor cells isolated from bone marrow, differentiation of the osteoprogenitor cells into osteoblasts and transplantation of the osteoblasts into the target sites in need of bone regeneration. However, all of the above-mentioned conventional techniques can be applied only for local bone regeneration and simply serve to fill an empty space of the bone, but suffer from disadvantages of difficulty to treat bone deficiency extensively distributed throughout the body, for example systemic bone deficiency due to osteoporosis and extensive bone deficiency due to osteonecrosis. Further, bone-absorption inhibitors used to treat osteoporosis have no bone regeneration-promoting ability and thereby suffer from many limitations in the treatment of extensive bone damage caused by osteoporosis.

In order to overcome the above-mentioned problems and disadvantages of conventional therapies, Korean Patent No. 0834718, assigned to the present applicant, discloses an osteogenesis-promoting cell composition for bone regeneration.

However, the above conventional art of the present applicant still suffers from a significant problem of a long-term culture period of cells.

More specifically, therapeutic methods using allografting, autografting or transplantation of topical autologous osteoblast-based therapeutic agents have been used for the treatment of local bone defects or necrosis. However, as discussed hereinbefore, the allograft technique suffers from the problems such as propagation risk of diseases, insufficient supply of transplants, possible immune responses, and a difficulty in complete regeneration of the transplants into self tissues. Meanwhile, the autograft technique solves or alleviates such problems suffered by the allografting, but has disadvantages such as a difficulty of securing donor sites, possible pathological episodes of the donor sites and the like. An autologous osteoblast-based cell therapeutic agent is a therapeutic method which was developed to solve the problems of these bone graft techniques, and it is known as a technique which is capable of achieving local bone regeneration by mass cultivation of osteoprogenitor cells isolated from bone marrow, differentiation of the osteoprogenitor cells into osteoblasts and transplantation of the thus-differentiated osteoblasts into the target lesions in need of bone regeneration. However, the autologous cell-based therapeutic approach is advantageous in terms of patient-specific therapy, but exhibits a variety of shortcomings such as expensiveness, complicated processes and long-term period of more than one month for production of therapeutic products, and consequently difficulty of immediate use of products on the spot where bone defects or fractures of patients are diagnosed.

In order to overcome disadvantages of bone regeneration by conventional bone transplantation techniques, development of cell-based therapeutic drugs has been accelerated, but production of such therapeutic drugs takes a long-term period of more than one month. Further, the autograft technique has disadvantages of limited donor sites and the allograft technique suffers from the risk associated with possible infection of diseases.

### DISCLOSURE

### Technical Problem

Therefore, the present invention has been made in view of the problems associated with conventional bone graft techniques as discussed above, and it is a first object of the present invention to provide a method for preparing a collagen gel composition for bone regeneration comprising collecting bone marrow from animal tissues and isolating nucleated cells from the bone marrow; and mixing the nucleated cells and a bio-matrix composed of type I collagen and apatite.

A second object of the present invention is to enhance osteoconductivity and cell affinity for osteoprogenitor cells or vascular cells, via use of a collagen-based matrix mixture.

A third object of the present invention is to provide an osteogenesis-promoting composition which is capable of facilitating bone regeneration by isolation of bone marrow-derived nucleated cells and co-transplantation of the nucleated cells and the matrix mixture into target sites in need of bone regeneration.

A fourth object of the present invention is to provide an injectable composition which is capable of simultaneously imparting bone filler and bone-regenerating cells, and a method of producing the same. For this purpose, a bone matrix mixture, including collagen which is capable of being mass-produced on an industrial scale, is mixed with bone marrow-derived nucleated cells of the patient and the mixture is then ready to use for the patient in need of bone regeneration within a short period of time.

A fifth object of the present invention is to achieve uniform delivery of the matrix composition and nucleated cells to target sites in need of bone formation irrespective of shape and morphology of bone defect lesions, by injection of an osteogenesis-promoting composition prepared according to the present invention into target lesions in need of local bone formation. Therefore, it is possible to treat a variety of fracture-related diseases.

A sixth object of the present invention is to provide a method for preparing a collagen gel composition for bone regeneration, which is suited for enhancing customer satisfaction via remarkably improved quality and reliability of the product.

### Technical Solution

In accordance with the present invention, the above and other objects can be accomplished by the provision of a method for preparing a collagen gel composition for bone regeneration comprising collecting bone marrow from animal tissues and isolating nucleated cells from the bone marrow; and mixing the nucleated cells and a bio-matrix composed of type I collagen and apatite.

### ADVANTAGEOUS EFFECTS

As illustrated hereinbefore, the present invention provides a method for preparing a collagen gel composition for bone regeneration comprising collecting bone marrow from animal tissues and isolating nucleated cells from the bone marrow; and mixing the nucleated cells and a bio-matrix composed of type I collagen and apatite.

Further, the present invention enhances osteoconductivity and cell affinity for osteoprogenitor cells or vascular cells, through use of a collagen-based matrix mixture.

Further, the present invention provides an osteogenesis-promoting composition which is capable of facilitating bone regeneration, by isolation of bone marrow-derived nucleated cells and co-transplantation of the nucleated cells and the matrix mixture into target sites in need of bone regeneration.

In addition, the present invention provides an injectable composition which is capable of simultaneously imparting bone filler and bone-regenerating cells, and a method of producing the same. For this purpose, a bone matrix mixture including collagen capable of being mass-produced is mixed with bone marrow-derived nucleated cells of the patient and the mixture is then rendered ready to use for the patient in need of bone regeneration within a short period of time.

Further, the present invention achieves uniform delivery of the matrix composition and nucleated cells to target sites in need of bone formation irrespective of shape and morphology of bone defect lesions, by injection of an osteogenesis-promoting composition prepared according to the present invention into target lesions in need of local bone formation. Therefore, it is possible to treat a variety of fracture-related diseases.

Finally, the present invention is significantly beneficial for enhancing customer satisfaction via remarkably improved quality and reliability of the product.

### DESCRIPTION OF DRAWINGS

FIG. 1 is a process flow chart illustrating a method for preparing a collagen gel composition for bone regeneration which is applied to the present invention;

FIG. 2 is a photograph of a nude mouse with scapular subcutaneous injection of 1 mL of a collagen gel composition for bone regeneration in accordance with the present invention;

FIG. 3 is an autoradiograph of a nude mouse taken 9 weeks after scapular subcutaneous injection of 1 mL of a collagen gel composition for bone regeneration in accordance with the present invention;

FIG. 4 is a photograph showing histological staining results of a nude mouse taken 9 weeks after scapular subcutaneous injection of 1 mL of a collagen gel composition for bone regeneration in accordance with the present invention;

FIG. 5 is an autoradiograph taken on Week 3 and 9 after injection of a collagen gel composition for bone regeneration in accordance with the present invention, following induction of a 10-mm long fracture in the rabbit forearm; and

FIG. 6 is an autoradiograph taken on Week 3 and 9 after injection of a collagen gel composition for bone regeneration in accordance with the present invention, following induction of a 15-mm long fracture in the rabbit forearm.

### BEST MODE

Hereinafter, the preferred embodiments of the present invention for accomplishing the above-mentioned objects and effects will be described in more detail with reference to the accompanying drawings.

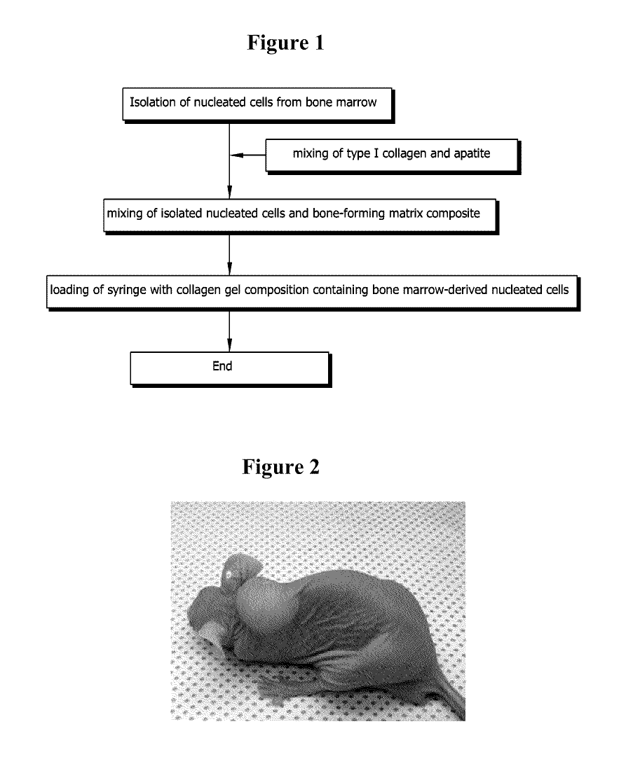

A method for preparing a collagen gel composition for bone regeneration, which is applied to the present invention, is constituted as shown in FIGS. 1 through 6.

In connection with description of the present invention hereinafter, if it is considered that description of known functions or constructions related to the present invention may make the subject matter of the present invention unclear, the detailed description thereof will be omitted.

Terms which will be described hereinafter are established taking into consideration functions in the present invention and may vary according to manufacturer's intention or a usual practice in the related art. Therefore, the terms used herein should be defined based on the context of the specification of the present invention.

As shown in FIG. 1, bone marrow is first harvested from animal tissues and nucleated cells are then isolated therefrom (Step of isolating nucleated cells).

Thereafter, the thus-isolated nucleated cells are mixed with a bio-matrix composed of type I collagen and apatite to thereby prepare a collagen gel composition for bone regeneration.

Apatite, a common calcium phosphate mineral, is a main source of phosphorous and is widely distributed in many igneous and metamorphic rocks. This compound may also be artificially synthesized. Apatite may be used as the source material for phosphate fertilizers, creams, toothpastes, artificial bones and materials thereof. When apatite is mixed with collagen for the above-mentioned applications, they undergo chemical reaction, which makes it possible to avoid long-term cell culture.

Preferably, the thus-prepared gel composition is placed and mixed in a syringe to which a connector or the like means is connected.

The cells obtained in the isolation step of nucleated cells consist of autologous nucleated cells.

The thus-isolated autologous nucleated cells are obtained by harvesting bone marrow with a size of 2 to 5 mm from animal bone marrow, followed by washing to isolate desired nucleated cells.

Further, the bio-matrix employs type I collagen free of terminal telopeptides thereof and apatite.

0.24 mL of type I collagen and 26.93 mg of apatite are added per 0.106 mL of a suspension of 1×10⁶ to 4×10⁶ nucleated cells having osteogenic capacity.

Hereinafter, preparation of a collagen gel composition for bone regeneration according to the present invention, as constituted above, will be illustrated.

### Mode for Invention

### Examples

Now, the present invention will be described in more detail with reference to the following examples. These examples are provided only for illustrating the present invention and should not be construed as limiting the scope and spirit of the present invention.

### Example 1

### Application of Bone-Regenerating Composition to Nude Mice

Bone marrow was collected from mouse tissues and nucleated cells were then isolated to prepare a cell suspension. Type I collagen and apatite were prepared as components for a bio-matrix.

The cell suspension, type I collagen and apatite were mixed to prepare 1 mL of a collagen gel composition for bone regeneration.

BALB/c nude mice (n=13, weighing about 23 g, irrespective of male and female) were given scapular subcutaneous injection of 1 mL of the collagen gel composition for bone regeneration.

On Week 3, 6 and 9 after injection of the bone-regenerating collagen gel composition, autoradiography and naked-eye examination were carried out followed by histological staining.

FIG. 2 shows a photograph of a nude mouse with scapular subcutaneous injection of 1 mL collagen gel composition for bone regeneration. It can be seen that the bone-regenerating collagen gel composition was normally injected into the target site as desired.

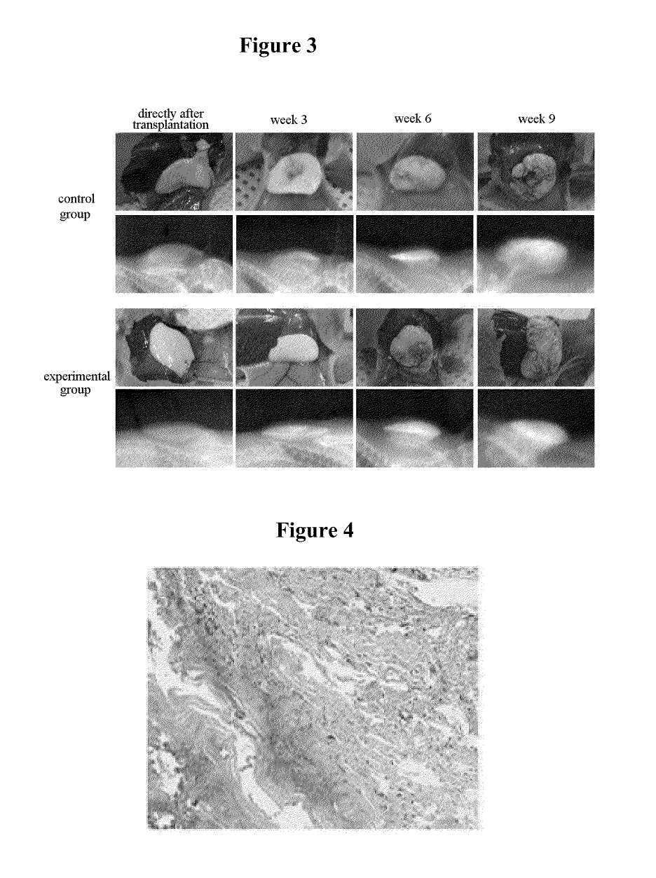

FIG. 3 is an autoradiograph of a nude mouse taken 9 weeks after scapular subcutaneous injection of 1 mL collagen gel composition for bone regeneration. As can be seen from FIG. 3, vascular formation was initiated by the cells which were externally introduced into the bone-regenerating collagen gel composition.

FIG. 4 shows the histological staining results of a nude mouse taken 9 weeks after scapular subcutaneous injection of 1 mL collagen gel composition for bone regeneration. As can be seen from FIG. 4, vascular formation and collagen formation were initiated by the cells which were externally introduced into the bone-regenerating collagen gel composition.

### Example 2

### Application of Bone-Regenerating Composition to an Animal Model with Induction of a 10-mm Long Fracture

Bone marrow was collected from rabbit tissues and nucleated cells were then isolated to prepare a cell suspension. Type I collagen and apatite were prepared as components for a bio-matrix.

The cell suspension, type I collagen and apatite were mixed to prepare 0.2 mL of a collagen gel composition for bone regeneration.

For this experiment, New Zealand white rabbits (n=7, weighing about 2.5 kg, irrespective of male and female) were assigned into an autograft control group (n=3) and an experimental group (n=4) for transplantation of a collagen gel composition containing bone marrow-derived nucleated cells.

According to Henry approach, the rabbit forearm was incised longitudinally to expose the radial shaft. Then, a 10-mm long bone defect of the radial shaft of the rabbit was created using a saw and the periosteum of the bone defect-induced lesion was thoroughly removed.

For the control group, the cancellous bone was previously collected from the ilium and bone transplantation was carried out on the bone-defective lesion, followed by suturing of skin and subcutaneous tissues.

For the experimental group, a cell composition containing bone marrow-derived nucleated cells was injected into a vacant space of the bone-defective lesion.

After autoradiography was conducted on Week 3, 6 and 9 of the experiment, scores were assigned according to a degree of bone union of the upper fractured portion, the lower fractured portion and the bone-defective region. The sum of the corresponding values was given to evaluate a degree of fracture union.

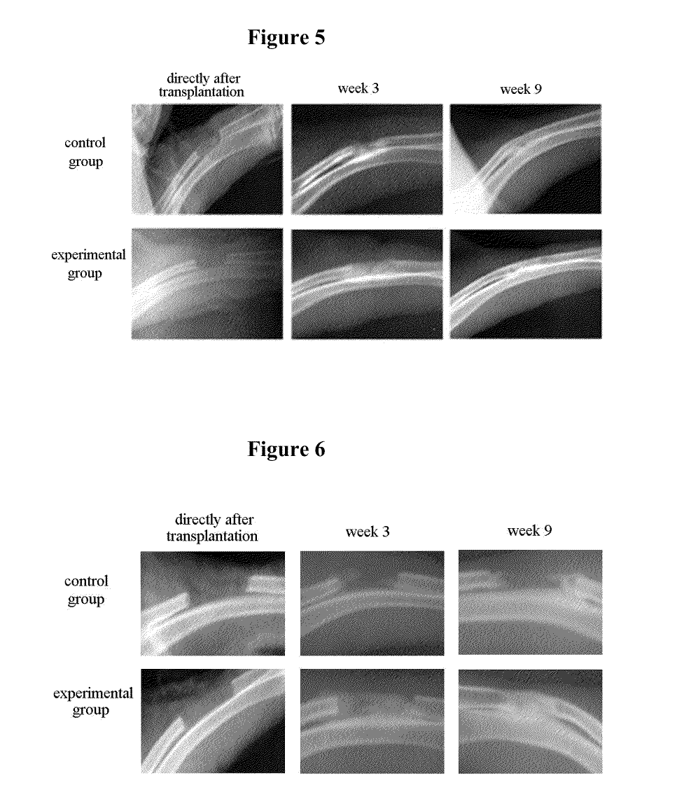

FIG. 5 shows an autoradiograph taken on Week 3 and 9 after injection of a collagen gel composition for bone regeneration, following induction of a 10-mm long fracture in the rabbit forearm. Two animal groups exhibited similar results in the bone formation.

### Example 3

### Application of Bone-Regenerating Composition to an Animal Model with Induction of a 15-mm Long Fracture

Bone marrow was collected from rabbit tissues and nucleated cells were then isolated to prepare a cell suspension. Type I collagen and apatite were prepared as components for a bio-matrix.

The cell suspension, type I collagen and apatite were mixed to prepare 0.2 mL of a collagen gel composition for bone regeneration.

For this experiment, New Zealand white rabbits (n=18, weighing about 2.5 kg, irrespective of male and female) were assigned into two groups, each consisting of 9 animals: a control group and an experimental group for transplantation of a collagen gel composition containing bone marrow-derived nucleated cells.

According to Henry approach, the rabbit forearm was incised longitudinally to expose the radial shaft. Then, a 15-mm long bone defect of the radial shaft of the rabbit was created using a saw and the periosteum of the bone defect-induced lesion was thoroughly removed.

For the control group, a bone-defective lesion was created in animals and washed with a 0.8% saline solution, followed by suturing of skin and subcutaneous tissues.

For the experimental group, a cell composition containing bone marrow-derived nucleated cells was injected into a vacant space of the bone-defective lesion.

After autoradiography was conducted on Week 3, 6 and 9 of the experiment, scores were assigned according to a degree of bone union of the upper fractured portion, the lower fractured portion and the bone-defective region. The sum of the corresponding values was given to evaluate a degree of fracture union.

FIG. 6 shows an autoradiograph taken on Week 3 and 9 after injection of a collagen gel composition for bone regeneration, following induction of a 15-mm long fracture in the rabbit forearm. It was confirmed that the experimental group exhibits significant bone formation, as compared to the control group.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A method for preparing a collagen gel composition for bone regeneration comprising:
collecting bone marrow from animal tissues and isolating nucleated cells from the bone marrow; and
mixing the nucleated cells and a bio-matrix composed of type I collagen and apatite.

2. The method according to claim 1, wherein the nucleated cells are autologous nucleated cells.

3. The method according to claim 2, wherein the isolated autologous nucleated cells are obtained by harvesting bone marrow with a size of 2 to 5 mm from animal bone marrow, and washing the bone marrow to isolate nucleated cells.

4. The method according to claim 1, wherein the bio-matrix includes terminal telopeptide-removed type I collagen and apatite.

5. The method according to claim 4, wherein 0.24 mL of type I collagen and 26.93 mg of apatite are added per 0.106 mL of a suspension of 1×10⁶ to 4×10⁶ nucleated cells having osteogenic capacity.
